(19) United States
(12) Patent Application Publication
(10) Pub. No.: US 2011/0245840 A1
(43) Pub. Date: Oct. 6, 2011

(54) INTRAOCULAR LENS INJECTOR SYSTEM

(76) Inventors: William J. Seyboth, Rochester, NY (US); Jon P. Cullen, Churchville, NY (US); Emin Engin, Rochester, NY (US); Christopher E. Wagner, Webster, NY (US); Graham W. Biddle, Ontario, NY (US)

(21) Appl. No.: 12/751,147

(22) Filed: Mar. 31, 2010

(51) Int. Cl. A61F 9/007

(52) U.S. Cl. 606/107

(57) ABSTRACT

An intraocular lens (IOL) assembly packaged for shipping, comprising an IOL injector component having a lumen wall, and an intraocular lens, at least one of the lumen wall and the optic comprising a first lens retention feature for impeding progress of the lens through said lumen toward the distal end. An IOL injector for injecting an IOL, comprising at least two projections extending from said lumen wall, the projections being configured and arranged to interfere with said soft tip prior to and during engagement of said soft tip with the IOL. An intraocular lens storage system, comprising a receptacle within the container, and a tapered portion, the tapered portion and the receptacle permitting a user's fingers to extend into the container to grasp a portion of a shuttle in the receptacle and remove the shuttle form the container, the receptacle defining a volume in which liquid is confined, the volume of the receptacle being less than the volume of the container.

### FIELD OF INVENTION

The present invention relates to intraocular lens injector systems.

### BACKGROUND OF THE INVENTION

Intraocular lenses (referred to herein as IOLs or, simply, lenses) are artificial lenses used to replace natural crystalline lenses of patients' when their natural lenses are diseased or otherwise impaired. Under some circumstances a natural lens may remain in a patient's eye together with an implanted IOL. IOLs may be placed in either the posterior chamber or the anterior chamber of an eye.

IOLs come in a variety of configurations and materials. Various instruments and methods for implanting such IOLs in an eye are known. Typically, an incision is made in a patient's cornea and an IOL is inserted into the eye through the incision. In one technique, a surgeon uses surgical forceps to grasp the IOL and insert it through the incision into the eye. While this technique is still practiced today, more and more surgeons are using IOL injectors, which offer advantages such as affording a surgeon more control when inserting an IOL into an eye and permitting insertion of IOLs through smaller incisions. Relatively small incision sizes (e.g., less than about 3 mm) are preferred over relatively large incisions (e.g., about 3.2 to 5+mm) since smaller incisions have been attributed with reduced post-surgical healing time and reduced complications such as induced astigmatism.

In order for an IOL to fit through a small incision, it is typically folded and/or compressed prior to entering the eye where it will assume its original unfolded/uncompressed shape. Since IOLs are very small and delicate articles of manufacture, great care is taken in their handling, both as they are loaded into an injector and as the lenses are injected into patients' eyes.

It is desirable that an IOL be expelled from the tip of the IOL injector and into the eye in an undamaged condition and in a predictable orientation. Should an IOL be damaged or expelled from the injector in an incorrect orientation, a surgeon may need to remove or further manipulate the IOL in the eye, possibly resulting in trauma to the surrounding tissues of the eye. To achieve proper delivery of an IOL, consistent loading of the IOL into the injector device, consistent engagement of the lens by the plunger tip and controlled movement of the lens through the injector lumen and into an eye with a limited opportunity for misalignment or damaging of the IOL is desirable.

Various IOL injectors and other devices have been proposed and produced which attempt to address issues related to ejecting IOLs into an eye, yet there remains a need for IOL injector components that facilitate surgical delivery of an IOL into an eye.

### SUMMARY

A first aspect of the invention is directed to an intraocular lens (IOL) assembly adapted to be attached to an injector, the assembly being packaged for shipping. The assembly comprises an IOL injector component having a lumen wall defining a lumen, the injector component having a longitudinal axis, and a proximal end and a distal end; and an intraocular lens comprising an optic body disposed in the lumen. At least one of the lumen wall and the optic body comprises a first lens retention feature, the first lens retention feature disposed such that the lens and the lumen wall interfere with one another thereby impeding progress of the lens through said lumen toward the distal end.

A second aspect of the invention is directed to an IOL injector for injecting an IOL. The injector comprises an injector body having a longitudinal axis and configured to maintain the IOL in an unstressed state having (i) a lumen wall defining a lumen extending therethrough and (ii) at least two projections extending from said lumen wall; and a plunger having a soft tip. The projections are configured and arranged to interfere with said soft tip prior to and during engagement of said soft tip with the IOL.

A third aspect of the invention is directed to an intraocular lens storage system, comprising a container having an amount of liquid disposed therein and a container open end, the open end having a length; and an IOL disposed in a shuttle, the shuttle residing in a receptacle within the container and having a receptacle open end. The IOL is immersed in the liquid. The container has a tapered portion extending at least a portion of the distance from the container open end to the receptacle open end such that a length of said receptacle open end is less than the length of the container open end and such that a user's fingers can extend into the container to grasp a portion of the shuttle and remove the shuttle form the container. The tapered portion and the receptacle define a volume in which the liquid is confined, the volume being less than the volume of the container.

### BRIEF DESCRIPTION OF THE DRAWINGS

Illustrative, non-limiting embodiments of the present invention will be described by way of example with reference to the accompanying drawings, in which the same reference number is used to designate the same or similar components in different figures, and in which:

FIG. 1 is a schematic illustration of an example of an injector system according to aspects of the present invention;

FIG. 2A illustrates an end view of the shuttle and lens of FIG. 1, which constitute an example of an IOL assembly according to aspects of the present invention;

FIG. 2B illustrates a top view of the shuttle and lens of FIG. 1 with the top portion of the shuttle omitted;

FIG. 2C illustrates a top view of an alternative embodiment of a shuttle and lens with the top portion of the shuttle omitted;

FIG. 3A is a partial cutaway, schematic illustration of the injector body, lens assembly and plunger of FIG. 1;

FIG. 3B is a cutaway schematic illustration of the injector body showing projections for centering the plunger;

FIG. 3C is a schematic illustration of the injector body comprising six projections for centering a plunger;

FIG. 3D is a side, cutaway, schematic illustration of the injector body, lens assembly and plunger of FIG. 1 showing centration of the plunger at a distal location by projections and at a proximal location by an end cap;

FIG. 3E is a top, cutaway, schematic illustration of the injector body, lens assembly and plunger of FIG. 1 showing the plunger actuated to a point where the lens is about to be compressed by the walls of the cartridge;

FIG. 4A is a cutaway, side, schematic illustration of the lens storage system of FIG. 1 (without the cover); and

FIG. 4B is a top, schematic illustration of the lens storage system of FIG. 4A.

### DETAILED DESCRIPTION

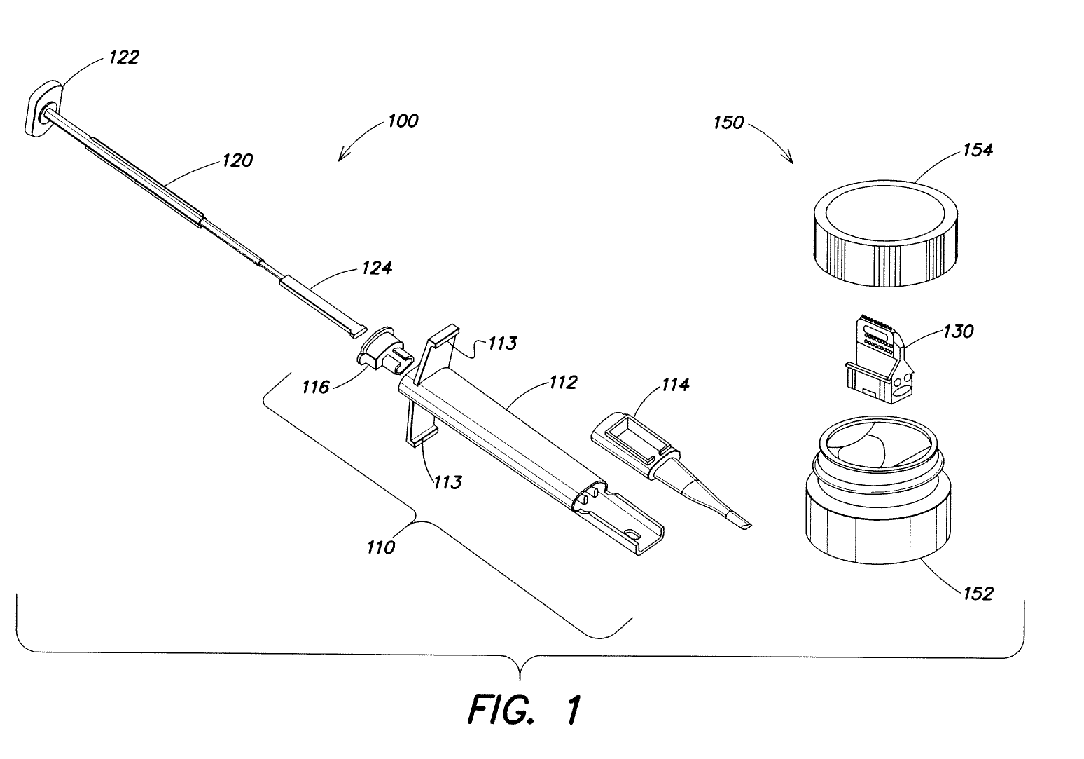

Aspects of the present invention are directed to various features of an intraocular lens injector system. An example of an injector system according to aspects of the present invention is shown in FIG. 1. System 100 comprises an injector body 110, a plunger 120 including a thumb press 122 and a soft tip 124, a shuttle 130 containing an IOL (not visible). Aspects of the invention are described below in greater detail with reference to components of the illustrated system. While an entire system is illustrated in FIG. 1, it is to be appreciated that the aspects of the invention described below need not be used in conjunction with all of the components of the system. Furthermore, the construction of components illustrated is by way of example and the design of components within the scope of the present invention may vary. For example, although the shuttle is maintained in a container 150 configured as a vial comprising a vial base 152 and a cover 154 coupled by threading, any suitable container may be used; and although the injector body is shown with a three-part construction (including (i) an injector body base 112 to which other injector body components are attached and which includes finger flanges 113 for facilitating actuation of the plunger 120, (ii) a cartridge 114 which receives the shuttle and compresses the lens upon actuation of the plunger, and (iii) an end cap 116 with which a proximal portion of the plunger interfaces), an injector body 110 may comprise one or more components.

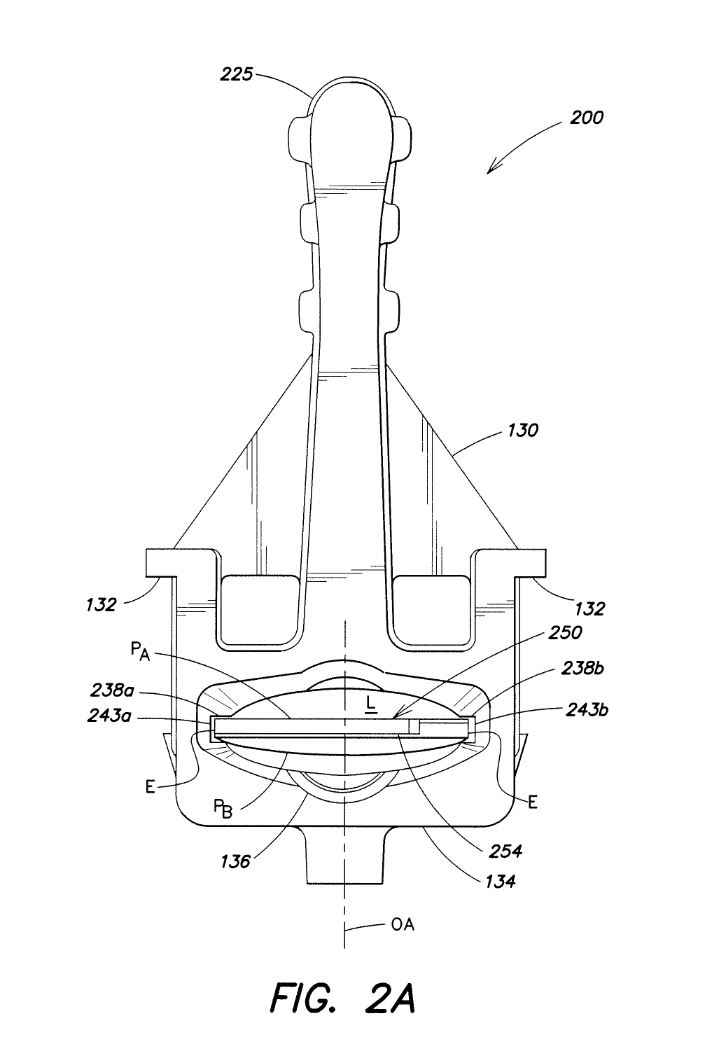

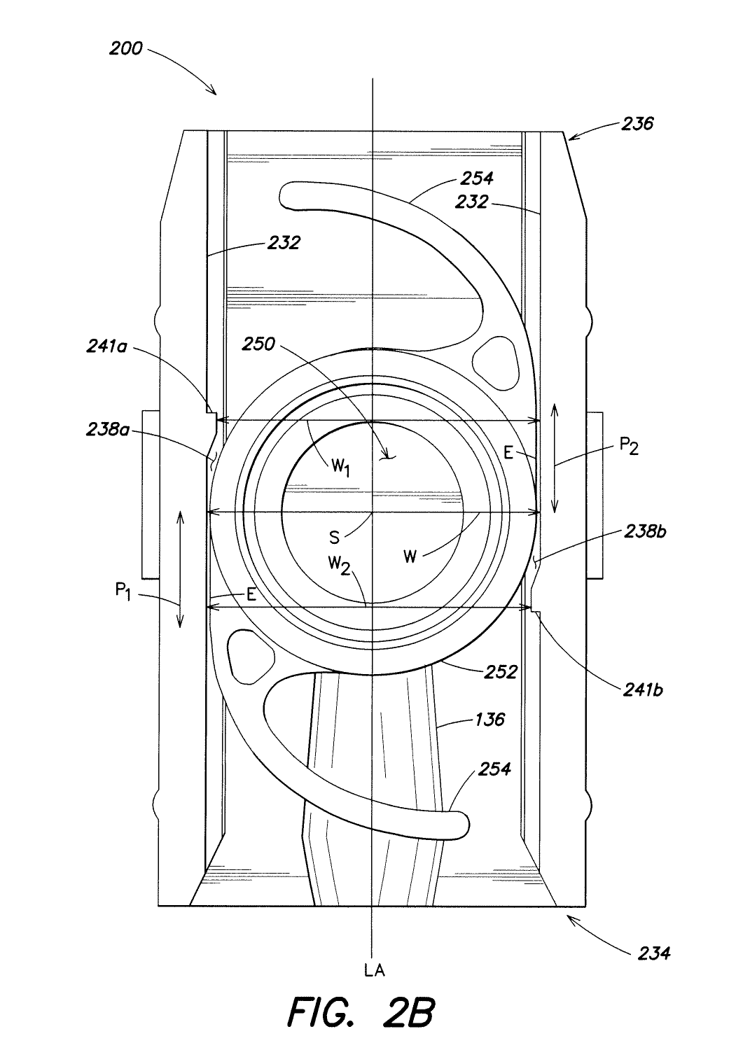

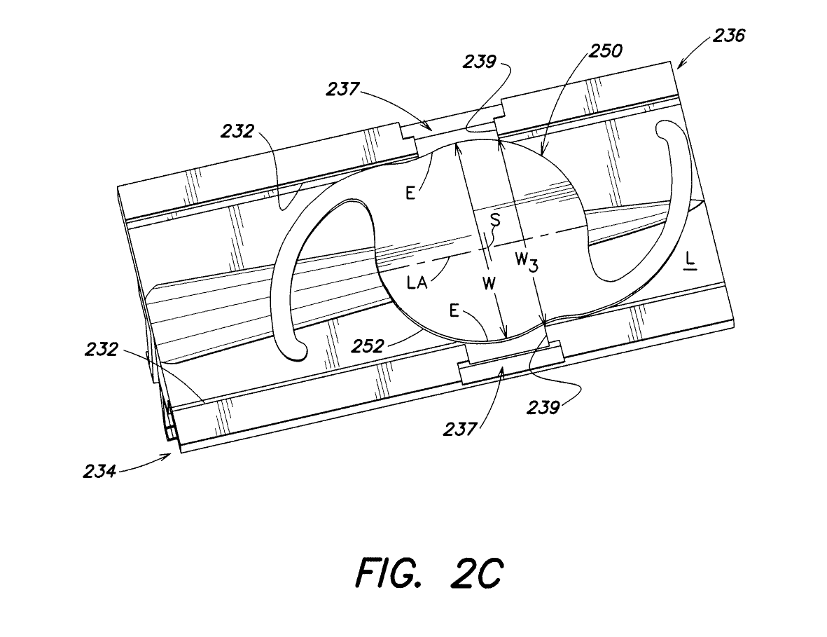

A first aspect of the invention is directed to an intraocular lens (IOL) assembly adapted to be attached to an injector, the assembly being packaged for shipping from a remote manufacturing or storage location to a surgical site. FIGS. 2A and 2B illustrate an example of an IOL assembly 200 according to aspects of the present invention. Assembly 200 comprises an IOL 250 and an injector component illustrated as shuttle 130. The packaging is omitted from FIGS. 2A and 2B to avoid obfuscation; however, one example of packaging is shown in FIG. 1 as a vial. It will be appreciated that packaging may include primary packaging to maintain the assembly in a sterile state and/or secondary packing which, for example, may be beneficial for commercial and/or logistical purposes. It will also be appreciated that separate packaging of such a lens assembly may be advantageous for inventory purposes where multiple lenses or types of lenses may be used with a single injector; accordingly, when packaged separately, an appropriate lens may be paired with an appropriate injector at a surgical site.

The IOL injector component has a lumen wall 232 defining a lumen L, a longitudinal axis LA, a proximal end 234 and a distal end 236. Intraocular lens 250 which comprises an optic body 252 is disposed in lumen L. Assembly 200 can be coupled to an intraocular lens injector body 110 (shown in FIG. 1) with the lumen of the assembly aligned with the lumen of the injector body 110, such that the lens can be moved through said lumens and into a patient's eye. In the illustrated embodiment, the lens will be compressed as it moves through the lumen which typically has a funnel shape. As discussed below, the lens haptics 254 can be managed to facilitate a consistent lens delivery. In particular, progress of the optic body can be impeded to permit one or more of the haptics to be positioned for safe delivery during compression.

In the illustrated embodiment, the lens has a location S along longitudinal axis LA at which the lens has a maximum width W as measured perpendicular longitudinal axis LA and the lumen wall comprises a protrusion constituting a first lens retention feature 241a. The protrusion is disposed distally of location S and defines a lumen width W₁ measured perpendicular to the longitudinal axis that is smaller than a diameter W of the optic body thereby impeding progress of the lens during acutuation of plunger 120 (shown in FIG. 1) through the lumen toward the distal end 236.

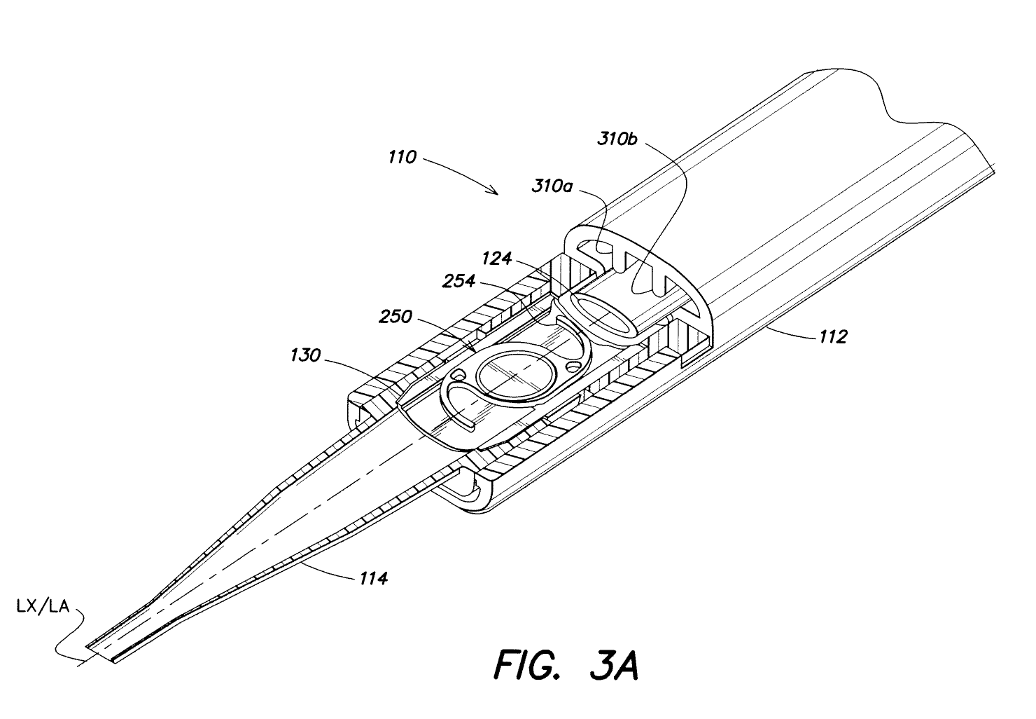

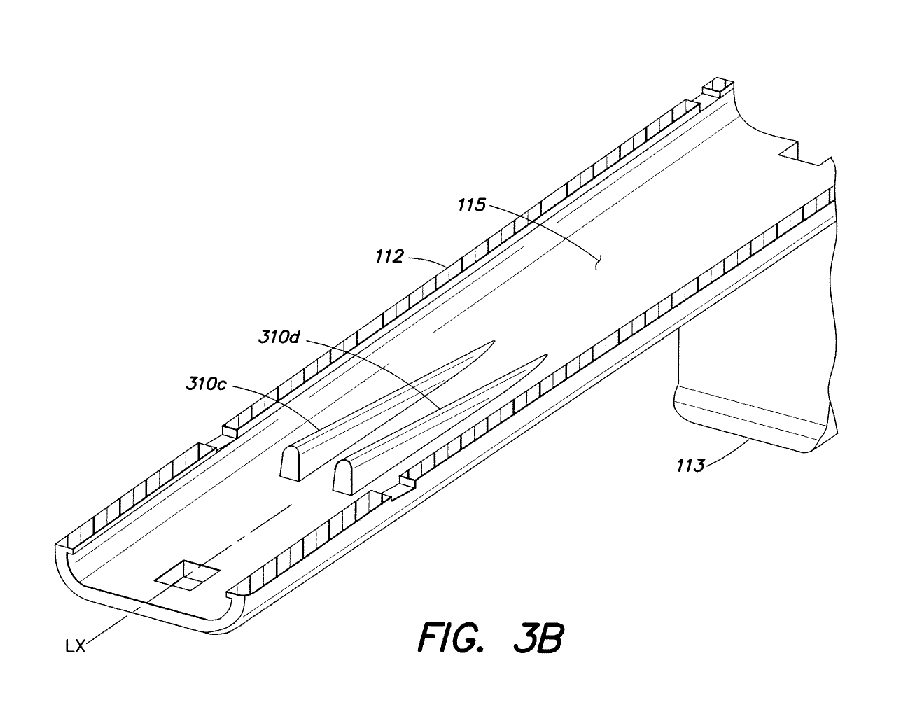

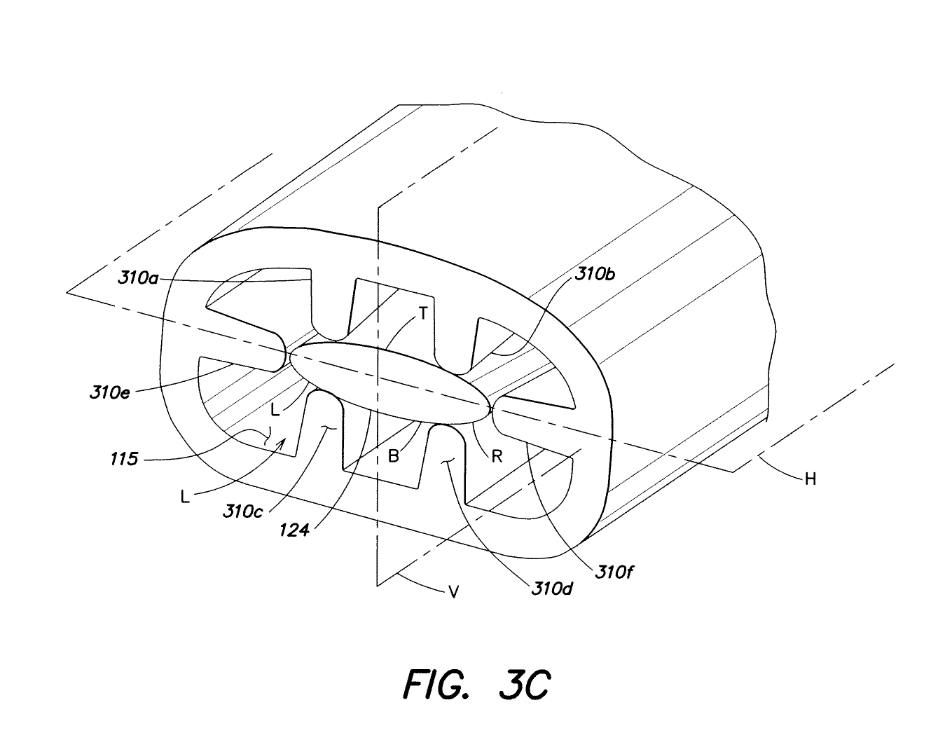

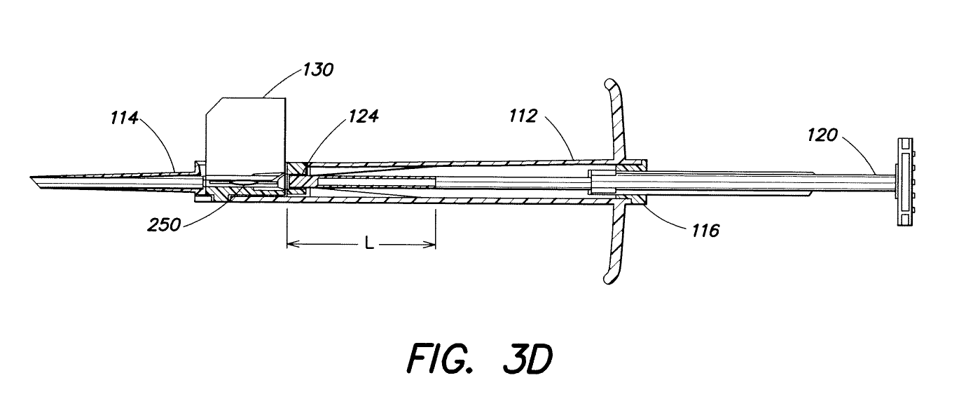

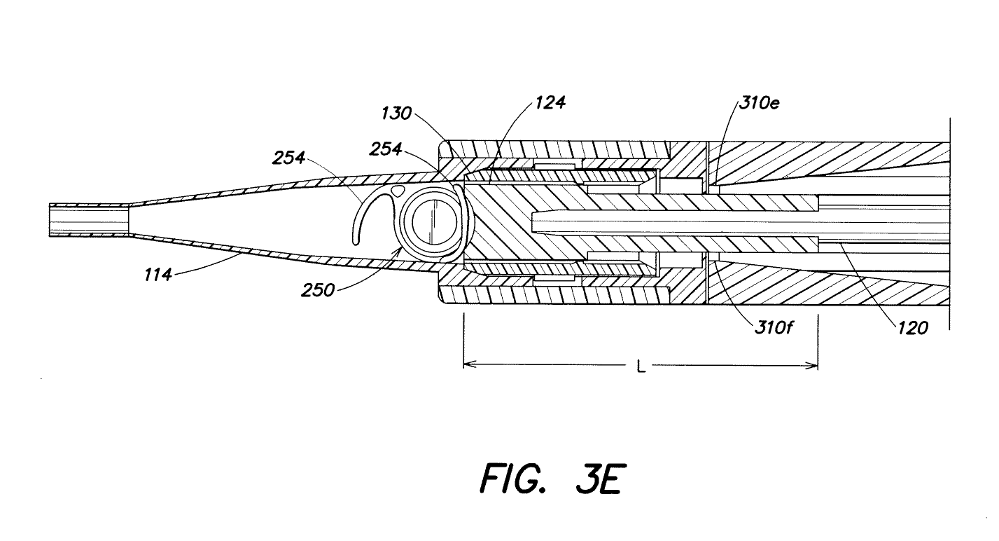

Although the illustrated embodiment shows a lens retention feature disposed on the lumen wall, it will be appreciated that to achieve benefits of the present invention, it is desirable that at least one of the lumen wall and the optic comprises a first lens retention feature, the first lens retention feature being disposed such that the lens and the lumen wall interfere with one another thereby impeding progress of the lens through lumen L toward the distal end 236. In some embodiment, for example, the lens comprises a protrusion (not shown) constituting the first lens retention feature to interfere with a feature (not shown) of the lumen wall thereby impeding progress of the lens toward the distal end. It will be appreciated that by impeding progress, engagement of a haptic 254 by plunger tip 124 will enable the haptic to be positioned proximate the optic body without substantial movement of the optic body through the lumen. As shown in FIG. 3E, in some embodiments, impediment to progress and flexibility of the haptics are selected such that the haptic can be moved on top of (or below) optic body 212 thereby decreasing the possibility of damaging the haptic during delivery of the lens into a patient's eye. It will also be appreciated that because the lens is made of a soft foldable material that, while progress is impeded to a degree, thereby allowing the haptics to be positioned on top of or below the lens, upon increased pressure from the plunger, the lens will fold or bend and move past the lens retention feature toward the distal end of the injector body.

In some embodiments of an intraocular lens (IOL) assembly packaged for shipping, the IOL injector component has a lumen wall 232 defining a lumen L, a longitudinal axis LA, a proximal end 234, a distal end 236, and an intraocular lens 250 which comprises an optic body 252 disposed in lumen L. However, the retention feature does not comprise a protrusion on either the lumen wall or the optic; rather, as shown in FIG. 2C, the lumen wall has a void 237 into which the edges E of the IOL extend such that an edge of the lumen wall forms retention feature 239. It will be appreciated that the void may extend the entire distance through the lumen wall (such that there is a hole in the wall) or only partway into the wall. In some such embodiment, the entire portion of the lumen that is located distally of the location S along longitudinal axis LA, at which the lens has a maximum width W as measured perpendicular longitudinal axis LA, has a width W₃ that is less than the maximum width W of the lens. Accordingly, an edge of the lumen wall forms a first lens retention feature 239 being disposed such that the optic body and the lumen wall interfere with one another thereby impeding progress of the lens through lumen L toward the distal end 236. Although the illustrated embodiment shows portions of the optic body extending into the voids, in other embodiments, portions of haptics or additional features of the lens (other than the optic body) (e.g., a feature extending form the optic body) may extend into the voids such that edges of the lumen wall are configured as lens retention features.

Referring again to FIGS. 2A and 2B, optionally, the lumen L further comprises opposing recesses 238a and 238b extending in a direction of longitudinal axis LA into which opposing optic edge portions E extend. The proximal end of the recesses are visible in FIG. 2A and the bottom halves of the recesses, which form shelves on which optic body edges E rest are visible in FIG. 2B. By locating the edges of the optic body in the recesses, orientation of the optic body can be controlled thereby contributing to the consistency of delivery of the lens into an eye. In the illustrated embodiment, first lens retention feature 241a is disposed in recess 238a; accordingly, the lens retention feature takes up a portion of the space in the recess thereby facilitating the retention feature in impeding the progress of the lens through the lumen.

As shown in FIG. 2B, in the illustrated embodiment, the opposing recesses extend in a direction substantially parallel to the longitudinal axis. For example, an insubstantial draft of approximately 2-3 degrees may be provided on lumen wall to facilitate the molding process. Alternatively, a substantial draft (e.g., greater than 10 degrees) may be provided which facilitates the lens compression process. As shown, the optic edge portions E extend parallel to the longitudinal axis over portions P₁ and P₂, and the lumen recesses comprise sidewalls 243a and 243b extending parallel to the edge portions in the direction of longitudinal axis LA. It will be appreciated that such a configuration increases the contact area between the edge E and the lumen wall thereby inhibiting lens rotation.

Referring to FIG. 3A, the plunger configuration further contributes to consistent delivery of the lens by the injector system. As illustrated in FIG. 3A, plunger 120 has a tip 124 configured and arranged to engage an at least one haptic 254 of the lens and, as described above, move the haptic toward the optic body while the lens is impeded by first lens retention feature 241a. Unlike conventional injectors, where the plunger is adapted such that the plunger tip engages a proximal edge of the optic, a system where the haptic is engaged contributes to haptic management by moving the haptics on top of (or below) the lens during lens compression. In the illustrated embodiment, the plunger tip is arranged to engage only one haptic. However, in other embodiments (not shown), for example in use with a lens having four haptics, the plunger is arranged to engage an at least two haptics of the lens. An example of such a lens is shown in U.S. Pat. No. 7,569,073, issued Aug. 4, 2009 to Vaudant et al.

In some embodiments, to facilitate engagement of the plunger tip with the haptics, the plunger tip is sized and shaped to substantially fill lumen L at a location along axis LA where the at least one haptic is disposed when the IOL is in an uncompressed state thereby ensuring haptic engagement. Although in the illustrated embodiment the tip fills the entire lumen, in some embodiments, the tip extends substantially the entire height of the lumen (i.e., in the direction extending parallel to the optical axis OA), however, the tip does not substantially fill the entire width of the lumen (i.e., in the direction transverse to the optical axis OA).

Referring again to FIG. 2B, in some embodiments, the lumen comprises a second lens retention feature 241b disposed proximally of the location of the maximum width S. The second lens retention feature is disposed on a surface of the lumen wall opposite a surface of the lumen wall on which first lens retention feature 241a is disposed. The second lens retention feature is configured and arranged to impede movement of the lens through said lumen toward the proximal end. The second lens retention feature defines a lumen width W₂ measured perpendicular to the longitudinal axis that is smaller than a diameter W of the optic body. It will be appreciated that such an arrangement further inhibits lens rotation during movement of the lens by the plunger beyond what can be achieved with a single lens retention feature 241a.

In some embodiments, it is advantageous that the lumen is sized to avoid contact with at least one of a portion P_{A} of the anterior surface and a portion P_{B} of the posterior surface of the optic. In some embodiments, the lumen is sized to avoid contact with, both, a portion P_{A} of the anterior surface and a portion P_{B} of the posterior surface of the optic. Such a configuration may be advantageous in decreasing resistance to lens advancement during movement of the lens by the plunger during compression and delivery of the lens into an eye.

Shuttle 130 has a textured handle 225 by which the shuttle can be grasped and IOL 250 moved without direct contact with the IOL. Surfaces 132 are arranged to permit the shuttle to be located in the injector body 110 (shown in FIG. 1) without the bottom surface 134 contacting the injector body. An access feature 136 facilitates an IOL being located into the shuttle using forceps, during assembly of a lens assembly.

Another aspect of invention is directed to plunger stability and the feel that is provided to an operator of the injector as the plunger is depressed and the IOL is moved through the lumen and into a patient's eye. It will be appreciated that stable movement along the injector longitudinal axis LX and consistent resistance to movement may provide control of delivery of the IOL. The present aspect of the invention will be discussed with reference to FIGS. 3A-3E.

FIGS. 3A-3E, illustrate an injector comprising an injector body 110 having a longitudinal axis LX and configured to maintain IOL 250 in an unstressed state. As indicated above, injector body 110 has a lumen wall 232 defining a lumen L extending therethrough. The injector body comprises projections 310a-310f extending from the lumen wall. The projections are configured and arranged to interfere with soft tip 124 prior to and during engagement of said soft tip with IOL 250. It will be appreciated that, because of the soft material, the projections will press into (i.e., indent) the soft tip thereby providing resistance to plunger moving forward and contributing to the feel provided to the operator.

According to the present aspect of the invention, there are at least two projections extending from said lumen wall (e.g., one from a top surface of the lumen wall and one form a bottom surface of the lumen wall), and the plunger comprises a soft tip. The portion of the tip over which the projections contact tip 124 may be selected to achieve a particular resistance to movement of the plunger. As shown in FIG. 3B, in some embodiments, the projections are tapered in the direction of the longitudinal axis with the height of the projections increasing in the distal direction such that tip is guided to a position in which is it is aligned with the longitudinal axis LA of the lens assembly prior to contact with the IOL. In some embodiments, projections 310e and 310f may extend form the lateral surfaces of the lumen wall thereby centering the plunger tip from side to side.

As illustrated in FIGS. 3D and 3E, to maintain the feel along the stroke of the plunger (i.e., from engagement with the IOL until compression begins) the tip may have a length extending a length L along the plunger shaft that is longer than is conventionally known. In some embodiments, tip 124 and projections 310 are configured and arranged such that contact with the highest point on the projections (measured from the lumen wall from which a projection extends) makes contact with the plunger soft tip at least from a longitudinal position prior to contact with IOL 250 until the IOL begins compression (e.g., where the lumen walls contact opposing edges of the optic body and begin funneling inward to cause compression of the lens). It will be appreciated that, after compression of the lens begins, the plunger will encounter resistance to advancement due to the compressing lens and resistance between projections and soft tip may not be necessary or desirable.

In some embodiments, the multiple projections can be positioned to provide enhanced stability of the plunger. For example, there may be two, three, four or more projections. In the illustrated embodiment, four projections 310a-310d extend from the lumen walls such that the soft tip can be divided into quadrants using imaginary planes as follows. The soft tip has a first plane V extending through the center of the distal end dividing the tip into a right portion R and left portion L and a second plane H extending through the center of the distal end dividing the tip into a top portion T and bottom portion B. In the illustrated embodiment, two of said projections contact the top portion, one on the left portion and one on the right portion, and two of said projections contacting the top portion, one on the left portion and one on the right portion. Also in the illustrated embodiment, projections 310e and 310f contact the sides of the plunger tip for lateral control.

The plunger may be further stabilized if the injector body and the plunger shaft are configured and arranged such that they contact one another at a location proximal to and separated from the projections. In some embodiments, the location of proximal contact is substantially at the proximal end 320 of the injector body. As shown in FIG. 3D, in the present embodiment, end cap 116 provides the proximal contact.

In some embodiments, the distal end of the soft tip has oval shaped perimeter and is concave. However, the tip may have any suitable perimeter shape (e.g., a circular, square, round, pentagonal or hexagonal) and any suitable end shape (e.g., flat or convex).

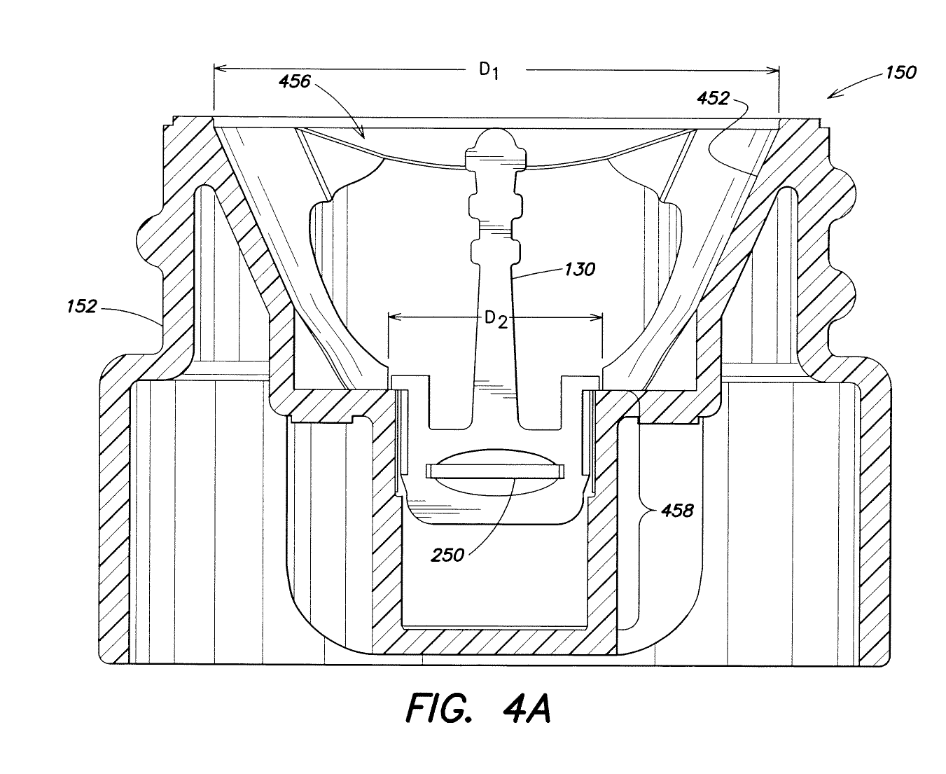

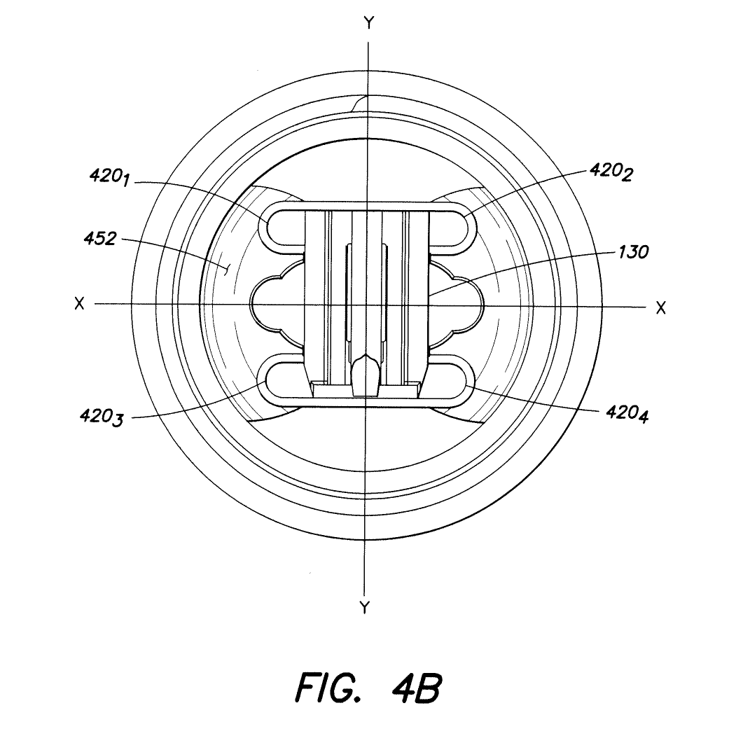

Other aspects of the invention are directed to a lens storage system facilitating storage and loading of an IOL. These aspects will be described with reference to FIGS. 4A and 4B. The lens storage system comprises a container 150 constituting primary packaging for the IOL, lens shuttle 130 for transporting the IOL between the container and an injector such as an injector described above.

Container 150 comprises base 152 which has an amount of liquid (e.g., saline solution) (not shown) disposed therein. The container has an open end 456 through which the liquid and IOL are introduced and removed. The open end has a length D₁ (e.g., a diameter).

IOL 250 is disposed in a shuttle 230 within the container. The applicants have found it advantageous that the container be easily manipulated while providing access to the shuttle to facilitate shuttle (and IOL) removal. An additional advantage of the present invention is that the above design goals are achieved while limiting the volume liquid needed to ensure that the IOL is maintained in a state of immersion in the liquid regardless of orientation of container 150.

Accordingly, the shuttle resides in a receptacle 458 within the container, the shuttle having a volume that is substantially less than the volume of the container. The container has a tapered portion 452 extending from said open end 456 to said receptacle open end such that a length D₂ of said receptacle open end is less than said length of the open end 456. It will be appreciated that the tapered portion need only extend a portion of the distance from the container open end to the receptacle open end, but may extend the entire distance or substantially the entire distance. Additionally, tapered portion 452 and container 150 are configured such that a user's fingers can extend into said container through open end 456 to grasp a portion of shuttle 130 (e.g., using handle 225 connected to the shuttle) and remove the shuttle form the container.

It will be appreciated that the tapered portion and the receptacle define a volume in which the liquid is confined, the volume being less than the volume of the container. The tapered portion is sized and shaped to permit fingers (e.g., a thumb and forefinger) to enter container 150 yet the amount of fluid present is relatively small. Although portion 452 is referred to as a tapered portion, it is to be appreciated that said portion need not be reduced in all cross sections. For example, in the illustrated embodiment, the tapered portion is tapered along cross-section X-X but not along cross-section YY.

An amount of liquid is present in the receptacle such that the IOL is immersed in the liquid. It will be appreciated that one or more channels 420₁-420₄ may be provided in the container to facilitate movement of the fluid, such that the IOL is immersed in the liquid regardless of orientation of the container. As discussed above, in some embodiments, shuttle 130 comprises a portion of an IOL injector. In such embodiments, the IOL can be removed from the container and loaded into injector without direct contact with the IOL and, after loading, the IOL is ready for injection merely by actuating plunger 120 (shown in FIG. 1).

Having thus described the inventive concepts and a number of exemplary embodiments, it will be apparent to those skilled in the art that the invention may be implemented in various ways, and that modifications and improvements will readily occur to such persons. Thus, the embodiments are not intended to be limiting and presented by way of example only. The invention is limited only as required by the following claims and equivalents thereto.

What is claimed is:

1. An intraocular lens (IOL) assembly adapted to be attached to an injector, the assembly being packaged for shipping, comprising:
an IOL injector component having a lumen wall defining a lumen, the injector component having a longitudinal axis, and a proximal end and a distal end; and
an intraocular lens comprising an optic body disposed in the lumen,
at least one of the lumen wall and the optic body comprising a first lens retention feature, the first lens retention feature disposed such that the lens and the lumen wall interfere with one another thereby impeding progress of the lens through said lumen toward the distal end.

2. The assembly of claim 1, wherein the lens has a location of maximum width as measured perpendicular the longitudinal axis and the lumen wall comprises a protrusion constituting the first lens retention feature, the protrusion disposed distally of said location thereby impeding progress of the lens through the lumen toward the distal end.

3. The assembly of claim 1, wherein the lens comprises a protrusion constituting the first lens retention feature thereby impeding progress of the lens toward the distal end.

4. The assembly of claim 1, wherein the lumen wall has at least one void into which an edge of the optic body extends, such that an edge of the lumen wall forms the first lens retention feature.

5. The assembly of claim 1, wherein the lumen further comprises opposing recesses extending in the direction of the longitudinal axis into which opposing optic edge portions extend, the lens retention feature being disposed in one of the recesses.

6. The assembly of claim 5, wherein the opposing recesses extend in a direction substantially parallel to the longitudinal axis.

7. The assembly of claim 1, wherein the intraocular lens comprises a haptic, the assembly further comprising a plunger having a tip, the plunger configured and arranged to engage an at least one haptic of the lens and move the haptic toward the optic while the lens is impeded by the first lens retention feature.

8. The assembly of claim 7, wherein the plunger is arranged to engage only one haptic.

9. The assembly of claim 8, wherein the plunger tip is sized and shaped to substantially fill the lumen at a location where the at least one haptic is disposed.

10. The assembly of claim 7, wherein the plunger is arranged to engage an at least two haptics of the lens.

11. The assembly of claim 10, wherein the plunger tip is sized and shaped to substantially fill the lumen at a location where the at least two haptics are disposed.

12. The assembly of claim 5, wherein the optic edge portions extend parallel to the longitudinal axis and the recesses comprise sidewalls extending parallel to the edge portions.

13. The assembly of claim 1, wherein the lumen comprises a second lens retention feature disposed on a surface opposite a surface on which the first lens retention feature is disposed, the second lens retention feature thereby impeding movement of the lens through said lumen toward the proximal end.

14. The assembly of claim 1, wherein the lumen is sized to avoid contact with at least a portion of an anterior surface and a posterior surface of the optic.

15. An IOL injector for injecting an IOL, comprising:
an injector body having a longitudinal axis and configured to maintain the IOL in an unstressed state having (i) a lumen wall defining a lumen extending therethrough and (ii) at least two projections extending from said lumen wall; and
a plunger having a soft tip, the projections being configured and arranged to interfere with said soft tip prior to and during engagement of said soft tip with the IOL.

16. The injector of claim 15, wherein the projections are configured and arranged to interfere with the soft tip until the lens is advanced to a portion along the longitudinal axis at which compression of the lens occurs.

17. The injector of claim 15, wherein the plunger further comprises a plunger shaft, the injector body and the plunger being configured such that the injector body and the plunger shaft contact one another at a location proximal to and separated from the projections.

18. The injector of claim 17, wherein the location is a location that is substantially at the proximal end of the injector body.

19. The injector of claim 15, wherein the soft tip has a first plane extending through the center of the distal end of the soft tip dividing the soft tip into a right portion and a left portion and a second plane extending through the center of the distal end of the soft tip dividing the soft tip into a top portion and a bottom portion, and wherein the at least two projections comprises at least four projections,
two of said projections contacting the top portion, one on the left portion and one on the right portion and
two of said projections contacting the top portion, one on the left portion and one on the right portion.

20. An intraocular lens storage system, comprising:
a container having an amount of liquid disposed therein and a container open end, the open end having a length; and
an IOL disposed in a shuttle, the shuttle residing in a receptacle within the container and having a receptacle open end,
the IOL immersed in the liquid,
the container having a tapered portion extending at least a portion of the distance from the container open end to the receptacle open end such that a length of said receptacle open end is less than the length of the container open end and such that a user's fingers can extend into the container to grasp a portion of the shuttle and remove the shuttle form the container,
the tapered portion and the receptacle defining a volume in which the liquid is confined, the volume being less than the volume of the container.

21. The system of claim 20, wherein the shuttle comprises a portion of an IOL injector.
